(19) United States
(12) Patent Application Publication
(10) Pub. No.: US 2003/0180769 A1
(43) Pub. Date: Sep. 25, 2003

(54) Substituted 4,4-difluoro-4-bora-3A,4A-diaza-s-indacene compounds for 8-color DNA sequencing

(76) Inventor: Michael L. Metzker, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/358,478

(22) Filed: Feb. 5, 2003

Related U.S. Application Data
(60) Provisional application No. 60/355,456, filed on Feb. 5, 2002.

(51) Int. Cl. C12Q 1/68; C07H 21/04; C12P 19/34

(52) U.S. Cl. 435/6; 435/91.2; 536/25.32

(57) ABSTRACT

The present invention describes a method of 8-color sequencing. Specifically, the present invention is directed to sequencing a sense strand and an antisense strand of a double-stranded polynucleotide comprising forming eight polynucleotide products differentially labeled with eight characteristic fluorophores, wherein said eight fluorophores comprise a set; and identifying each of the eight polynucleotide products by a fluorescence or an absorption spectrum of the characteristic fluorophores.

### RELATED APPLICATIONS

This application claims priority to U.S. Provisional Application Ser. No. 60/355,456, filed Feb. 5, 2002, hereby incorporated by reference in its entirety.

### BACKGROUND OF THE INVENTION

1. Field of the Invention

The present invention relates to the fields of molecular biology, genetics and organic chemistry. The invention is directed to methods for simultaneous detection of forward and reverse sequencing reactions using a set of fluorophores for 8-color sequencing of polynucleotides. Compositions comprising the set of fluorophores are also provided.

2. Related Art

The ability to detect a polynucleotide and specific sequence of a polynucleotide is critical for understanding the function and control of genes and for diagnosing genetically-inherited diseases. Native DNA consists of two linear polymers or strands of nucleotides: a sense strand and an antisense strand. Each strand of DNA is a chain of nucleotides linked by phosphodiester bonds. The two strands are held together in an antiparallel orientation by hydrogen bonds between complementary bases of the nucleotides of the two strands: deoxyadenosine (A) pairs with thymidine (T) and deoxyguanosine (G) pairs with deoxycytidine (C). The development of the polymerase chain reaction (PCR) technique provided a significant advance in polynucleotide manipulation (see U.S. Pat. Nos. 4,683,195; 4,683,195; 4,800,159; and Saiki et al., (1985) Science 230:1350).

The development of reliable methods for sequence analysis of DNA (deoxyribonucleic acid) and RNA (ribonucleic acid) has been essential to the success of recombinant DNA and genetic engineering. Used with the other techniques of modern molecular biology, nucleic acid sequencing allows dissection of animal, plant and viral genomes into discrete genes with defined chemical structure. Because the function of a biological molecule is determined by its structure, defining the structure of a gene is crucial to the eventual useful manipulation of this basic unit of hereditary information. After a gene is isolated and characterized, it is modified to effect desired changes in their structure that allow the production of a gene product—a protein—with different properties than those possessed by the original gene product.

The development of modern nucleic acid sequencing methods involved parallel developments in a variety of techniques. One was the emergence of simple and reliable methods for cloning small to medium-sized strands of DNA into bacterial plasmids, bacteriophages, and small animal viruses. Cloning allowed the production of pure DNA in sufficient quantities to allow chemical analysis. Another was the use of gel electrophoretic methods for high resolution separation of oligonucleotides on the basis of size. The key development, however, was the introduction of methods of generating sets of cloned, purified DNA fragments that contain, in their collection of lengths, the information necessary to define the sequence of the nucleotides comprising the parent DNA molecules.

Holland et al. (1991) described an assay known as a Taqman® assay. The 5′→3′ exonuclease activity of Taq polymerase is employed in a polymerase chain reaction product detection system to generate a specific detectable signal concomitantly with amplification. An oligonucleotide probe, nonextendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′→3′ exonuclease activity of Taq polymerase degrades the probe into smaller fragments that can be differentiated from undegraded probe. The assay is sensitive and specific and provides a significant improvement over more cumbersome detection methods. A version of this assay is also described in Gelfand et al., in U.S. Pat. No. 5,210,015. U.S. Pat. No. 5,210,015 to Gelfand, et al., and Holland, et al., PNAS 88:7276-7280 (1991) are hereby incorporated by reference in their entirety.

Further, U.S. Pat. No. 5,491,063 to Fisher, et al., provides a Taqman®-type assay. The method of Fisher et al. provides a reaction that results in the cleavage of single-stranded oligonucleotide probes labeled with a light-emitting label wherein the reaction is carried out in the presence of a DNA binding compound that interacts with the label to modify the light emission of the label. The method utilizes the change in light emission of the labeled probe that results from degradation of the probe. The methods are applicable in general to assays that utilize a reaction that results in cleavage of oligonucleotide probes, and in particular, to homogeneous amplification/detection assays where hybridized probe is cleaved concomitantly with primer extension. A homogeneous amplification/detection assay is provided that allows the simultaneous detection of the accumulation of amplified target and the sequence-specific detection of the target sequence. U.S. Pat. No. 5,491,063 to Fisher, et al. is hereby incorporated by reference.

Presently there are several approaches to DNA sequence determination, see, e.g., the dideoxy chain termination method, Sanger et al., Proc. Natl. Acad. Sci., 74:5463-67 (1977); the chemical degradation method, Maxam et al., Proc. Natl. Acad. Sci., 74:560-564 (1977); and hybridization methods, Drmanac et al, Genomics, 4:114-28 (1989), Khrapko, FEB 256:118-22 (1989). The chain termination method has been improved in several ways and serves as the basis for all currently available automated DNA sequencing machines. See, e.g., Sanger et al., J. Mol. Biol., 143:161-78 (1980); Schreier et al., J. Mol. Biol., 129:169-72 (1979); Smith et al., Nucleic Acids Research, 13:2399-2412 (1985); Smith et al., Nature, 321:674-79 (1987) and U.S. Pat. No. 5,171,534; Prober et al., Science, 238:336-41 (1987); Section II, Meth. Enzymol., 155:51-334 (1987); Church et al., Science, 240:185-88 (1988); Swerdlow and Gesteland, Nucleic Acids Research, 18: 1415-19 (1989); Ruiz-Martinez et al., Anal. Chem., 2851-58 (1993); Studier, PNAS, 86:6917-21 (1989); Kieleczawa et al., Science, 258:1787-91; and Connell et al., Biotechniques, 5:342-348 (1987).

The method developed by Sanger is referred to as the dideoxy chain termination method. In a commonly-used variation of this method, a DNA segment is cloned into a single-stranded DNA phage, such as M13. These phage DNAs serve as templates for the primed synthesis of the complementary strand by conventional DNA polymerases. The primer is either a synthetic oligonucleotide or a restriction fragment isolated from the parental recombinant DNA that hybridizes specifically to a region of the M13 vector near the 3′ end of the cloned insert. In each of four sequencing reactions, the primed synthesis is carried out in the presence of enough of the dideoxy analog of one of four possible deoxynucleotides so that the growing chains are randomly terminated by the incorporation of 2′, 3′-dideoxynucleotides using DNA polymerase. The reaction also includes the natural 2′-deoxynucleotides, which extend the DNA chain by DNA synthesis. Thus, balanced appropriately, competition between chain extension and chain termination results in the generation of a set of nested DNA fragments, which are uniformly distributed over thousands of bases and differ in size as base pair increments. Electrophoresis is used to resolve the nested DNA fragments by their respective size. However, if the labels are attached to the primer, four primed syntheses are carried out in the presence of one dideoxy analog and all four possible deoxynucleotides so that the growing chains are uniformly terminated for the specific complement base by incorporation. The products from each of the four primed synthesis reactions are loaded into individual lanes and are separated by polyacrylamide gel electrophoresis. Radioactive label incorporated in the growing chains are used to develop an autoradiogram image of the pattern of the DNA in each electrophoresis lane. The sequence of the deoxynucleotides of a single strand in the cloned DNA is determined from an examination of the pattern of bands in the four lanes. Because the products from each of the four synthesis reactions must be run on separate gel lanes, problems arise with comparing band mobilities in the different lanes.

In general, automated DNA sequencing machines analyzes DNA fragments having different terminating bases that are labeled with different fluorescent dyes, which are attached either to a primer for dye-primer sequencing in which the fluorescent dyes are attached to the 5′ end of the primers (Smith et al. 1987), or to the base of the dideoxynucleotide for dye terminator sequencing in which the fluorescent dyes are attached to the C⁷ position of a purine terminating base and the C⁵ of a pyrimidine terminating base (Prober et al., 1987). In this case, a fluorescence detector is employed to detect the fluorophore-labeled DNA fragments. The four different dideoxy-terminated samples are run in four separate lanes or, if labeled differentially, in the same lane.

The method of Fung et al., U.S. Pat. No. 4,855,225, uses a set of four chromophores or fluorophores with different absorption or fluorescent maxima. Each of these tags is coupled chemically to the primer used to initiate the synthesis of the fragment strands. In turn, each tagged primer is then paired with one of the dideoxynucleotides and used in the primed synthesis reaction with conventional DNA polymerases. The labeled fragments are then combined and loaded onto the same gel column for electrophoretic separation. Base sequence is determined by analyzing the fluorescent signals emitted by the fragments as they pass a stationary detector during the separation process.

However, obtaining a set of dyes to label the different fragments is a major difficulty in automated DNA sequencing systems. First, it is difficult to find three or more dyes that do not have emission bands that overlap significantly, since the typical emission band half-width for organic fluorescent dyes is about 40-80 nanometers (nm) and the width of the visible spectrum is only about 350-400 nm. Second, even if dyes with non-overlapping emission bands are found, then the set often exhibits respective low fluorescent efficiencies and are unsuitable for DNA sequencing. Pringle et al. (1988) present data indicating that increased gel loading does not compensate for low fluorescent efficiencies.

Another difficulty with obtaining an appropriate set of dyes is that when several fluorescent dyes are used concurrently, excitation becomes difficult, because the absorption bands of the dyes are often widely separated. The most efficient excitation occurs when each dye is illuminated at the wavelength corresponding to its absorption band maximum. Thus, one often is forced to compromise either the sensitivity of the detection system or the increased cost of providing separate excitation sources for each dye. Additionally, as the number of differently sized fragments in a single column of a gel reaches greater than a few hundred, the physiochemical properties of the dyes and the means by which they are linked to the fragments become critical, because the charge, molecular weight, and conformation of the dyes and linkers must not affect adversely the electrophoretic mobilities of closely-sized fragments. Changes in electrophoretic mobility sometimes results in extensive band broadening or reversal of band positions on the gel, thereby destroying the correspondence between the order of bands and the order of the bases in the nucleic acid sequence. Due to the many problems associated with altered electrophoretic mobility, correction of mobility discrepancies by computer software is necessary in prior art systems. Finally, the fluorescent dyes must be compatible with the chemistry used to create or manipulate the fragments. For example, in the chain termination method the dyes used to label primers and/or the dideoxy chain terminators must not interfere with the activity of the polymerase or reverse transcriptase employed.

Because of these severe constraints, only a few sets of fluorescent dyes have been found that are useful in DNA sequencing, particularly automated DNA sequencing, and in other diagnostic and analytical techniques, e.g., Smith et al. (1985, cited above); (Prober et al., 1987); Hood et al., European patent application 8500960; Bergot et al. (cited above); Fung et al. (cited above); Connell et al. (cited above); Lee, et al., Nucleic Acids Research, 20:2471-83 (1992); and Menchen et al., U.S. Pat. No. 5,188,934. Dyes commonly used to correct for differences in gel mobility between different dye-labeled primers include fluorescein and its derivatives and rhodamine and its derivatives, cyanines, coumarins, sulfonated pyrenes, squaraines and alexas.

Custom sequencing primers have also been used and refer to any oligonucleotide sequence that acts as a suitable DNA sequencing primer. However, all custom sequencing primers must be coupled to a 5′-leader sequence (5′-CAGGA) and must use the M13RP1 mobility correction software to generate properly-spaced DNA termination fragments. U.S. Pat. No. 6,087,099 to Gupte et al. teaches a specially designed oligomer that contains a reverse complement sequence along with a standard primer that when used in PCR generates a double stranded DNA product that denatures into a single strand containing the sequence of both original strands. Thus, sequencing of the amplified single stranded DNA yields double stranded sequence information.

A class of dyes, 4,4-difluoro-4-bora-3A,4A-diaza-s-indacene BODIPY fluorophores has been described (Haugland, et al., Molecular Probes: Handbook of Fluorescent Probes and Research Chemicals, pp. 24-32, and U.S. Pat. No. 4,774,339). The parent heterocyclic molecule of the BODIPY fluorophore is a dipyrrometheneboron difluoride compound and which is modified to create a broad class of spectrally-discriminating fluorophore. The conventional naming of these dyes is BODIPY followed by their approximate absorption/emission maxima, i.e., BODIPY 530/550. BODIPY fluorophores have been utilized for a wide variety of uses, including high throughput fluorescence polarization assays (for example see, Banks et al., 2000), probing and labeling proteins, and variations including extending conjugation and restricting bond rotations to produce constrained dyes with longer absorption maxima (620-660 nm) and fluorescence maxima (630-680 nm) have been described (Chen et al., 2000).

Prior to the present invention, the availability of a set of fluorescent dyes that (1) are physiochemically similar; (2) permit detection of spatially overlapping target substances, such as closely spaced bands of DNA on a gel; (3) extend the number of bases that are determined on a single gel column by current methods of automated DNA sequencing; (4) are amenable for use with a wide range of preparative and manipulative techniques; and (5) are spectrally resolvable at eight different wavelengths, has not been described.

DNA sequencing assays predominantly employ a 4-color sequencing assay and are relegated to sequencing a single-strand of DNA, such as a sense strand, with four spectrally differentiated dyes in a first reaction followed by a second reaction using the same 4-color dyes to obtain sequence information of the complementary strand, such as in this case the antisense strand. The invention described herein provides a method to detect up to eight oligonucleotides, ribonucleotides, deoxynucleotides, or dideoxyribonucleotides, that are differentially-labeled with a fluorophore, wherein the fluorophore comprises a substituted 4,4-difluoro-4-bora-3A,4A-diaza-s-indacene (BODIPY fluorophore) compound. It is known in the art that BODIPY fluorophores have improved spectral characteristics, narrower band width, insensitivity to solvent or pH, and improved photostability compared to conventional fluorescein and rhodamine dyes. U.S. Pat. Nos. 5,614,386; 5,861,287; and 5,994,063 are incorporated by reference in their entirety. As described herein, the new substituted 4,4-difluoro-4-bora-3A,4A-diaza-s-indacenes (BODIPY fluorophores) provide a bathochromic shift, as compared to previously described BODIPY fluorophores, and an unexpected improvement in the spectral resolution such that a set of eight spectrally resolvable compounds useful for a simultaneous detection of forward and reverse DNA sequencing reactions are provided.

### SUMMARY OF THE INVENTION

In the present invention there is a method of sequencing a sense strand and an antisense strand of a double-stranded polynucleotide comprising comprising i) denaturing the double-stranded polynucleotide to provide the sense strand and the antisense strand; ii) reacting the sense strand with a first set of four differentially labeled polynucleotides; iii) reacting the antisense strand with a second set of four differentially labeled polynucleotides; iv) identifying each of the eight polynucleotides by a fluorescence or an absorption spectrum of the fluorophore; and v) determining the sequence of the sense strand from the polynucleotides differentially labeled with the first set of fluorophores and the sequence of the antisense strand from the polynucleotides differentially labeled with the second set of fluorophores.

Another embodiment of the invention is a method of 8-color sequencing of a polynucleotide comprising the steps of i) forming eight classes of polynucleotides wherein each class of polynucleotides is labeled with a fluorophore and each fluorophore is different; ii) electrophoretically separating the classes of polynucleotides; iii) illuminating the separated polynucleotides with a wavelength capable of causing the fluorophores to fluoresce; and iv) identifying the classes of polynucleotides by the fluorescence or absorption spectrum of the fluorophores. In a specific embodiment, the fluorophore is at least one BODIPY fluorophore that has been chemically modified. In one embodiment, the polynucleotides are separated in at least one lane of the gel. In another embodiment, the eight fluorophores are linked to the 5′ ends of the polynucleotides, or the 3′ ends of the polynucleotides.

A method of distinguishing polynucleotides having different 3′-terminal dideoxynucleotides in a chain termination method of DNA sequencing, the method comprising the steps of: i) forming eighth classes of polynucleotides by extending from primers a plurality of polynucleotides by means of a DNA polymerase or a reverse transcriptase in the presence of a dideoxyadenosine triphosphate, a dideoxycytosine triphosphate, a dideoxyguanosine triphosphate, and a dideoxythymidine triphosphate, and wherein the eight classes of polynucleotides are labeled at a 5′ position with a different fluorophore; ii) clectrophoretically separating the classes of polynucleotides; iii) illuminating the separated polynucleotides with a wavelength capable of causing the fluorophores to fluoresce; and iv) identifying the classes of polynucleotides in the bands by the fluorescence or absorption spectrum of the fluorophores.

Another specific embodiment of the invention is a method for distinguishing polynucleotides having different ribonucleotides in a method of labeling polynucleotides by enzymatic incorporation, said method comprising the steps of i) forming a mixture of four classes of polynucleotides, the four classes comprising polynucleotides having different terminal nucleotide triphosphates, wherein said triphosphates are linked to a BODIPY fluorphore that contains at least one reactive functional group; and wherein said BODIPY fluorophores comprise a first set and all are different; ii) forming a second mixture of four classes of polynucleotides, the four classes comprising polynucleotides having different terminal nucleotide triphosphates; wherein said triphosphates are linked to a BODIPY fluorphore that contains at least one reactive functional group; and wherein said BODIPY fluorophores comprise a second set and all are different, and are different that the first set; iii) electrophoretically separating the polynucleotides in the first mixture and the second mixture; iv) illuminating the first and second mixtures with a wavelength capable of causing the fluorophores to fluoresce; and v) identifying the classes of polynucleotides in the bands by the fluorescence or absorption spectrum of the fluorophores.

In further specific embodiments, the terminal nucleotide triphosphates are adenosine triphosphate, guanosine triphosphate, cytidine triphosphate, and uridine triphosphate. Embodiments are also contemplated in which the terminal nucleotide triphosphates are deoxyadenosine triphosphate, deoxyguanosine triphosphate, deoxycytidine triphosphate, and deoxythymidine triphosphate. In yet further embodiments, the terminal nucleotide triphosphates are dideoxyadenosine triphosphate, dideoxyguanosine triphosphate, dideoxycytidine triphosphate, and dideoxythymidine triphosphate.

Also provided is a method of labeling a nucleic acid for 8-color sequencing comprising the steps of i) forming an plurality of oligonucleotides substituted with at least two fluorophores comprising a donor and an acceptor; wherein said oligonucleotides are separated eight classes, wherein said eight donor fluorophores comprise a donor set and are the same or different; and wherein eight acceptor fluorophores comprise an acceptor set and are all different; ii) annealing said oligonucleotide classes to a strand of a polymerase chain reaction product to generate a substrate for a 5′ to 3′ exonuclease activity; iii) amplifying said oligonucleotide classes, wherein said exonuclease activity degrades said oligonucleotides, wherein said donor is released; and iv) detecting said oligonucleotide classes. In other embodiments, it is envisioned that the acceptor fluorophore is different in all eight classes of oligonucleotides and the donor fluorophore is different or the same.

In all of the above embodiments, a specific embodiment is contemplated whereby that each BODIPY fluorophore in the set is coupled to the primer suitable for sequencing by a linker.

Also, in all of the above embodiments, the DNA polymerase may be Thermosequenase, AmpliTaqFS, Klenow fragment, SEQUENASE® DNA polymerase, Bst DNA polymerase, AMPLITAQ® DNA polymerase, Pfu (exo-)DNA polymerase, rTth DNA polymerase or Vent(exo-) DNA polymerase. In other specific embodiments, the reverse transcriptase is AMV-RT, M-MuLV-RT or SuperScript RT®. In a specific embodiment, the sequencing is performed by an automated DNA sequencing instrument.

In all the above specific embodiments, the fluorophores may comprise at least one BODIPY fluorophore selected from the group consisting of BODIPY 542/563, BODIPY B410, BODIPY B411, BODIPY 503/512, BODIPY 523/547, BODIPY 581/591, BODIPY 630/650 and BODIPY 650/665. In further specific embodiments, the fluorphores may also comprises fluoresceins, rhodamines, cyanines, coumarins, sulfonated pyrenes, squaraines or alexas.

In each of the above embodiments, each BODIPY fluorophore exhibits a characteristic adsorption maxima that is spectrally resolved as compared to the other BODIPY fluorophores in the set, and the adsorption maxima is in the range of about 500 to about 700 nm. Further, the designation of, for example, the eighth class having a terminal dideoxythymidine is not meant to be limiting the scope of the eight reactions, in that a sixth class of polynucleotides have a terminal dideoxythymidine provided that the eighth class has a terminal dideoxycytidine. Thus, as long as the four dideoxy analogs are present, the exact class designations are not limiting.

In other specific embodiments, each BODIPY fluorophore is attached at the 5′ end of the products of the sequencing reaction and an additional fluorophore is attached at a 3′ position of the product of the sequencing reaction or at one or more internal positions of the products of the sequencing reaction. In a preferred specific embodiments, the additional fluorophore has an adsorption maxima of about 500 to about 700 nm and an emission maxima of about 500 to about 700 nm.

The present invention also provides as a compositions of matter, a 4,4-difluoro-5,7-dimethyl-4-bora-3a,4a-diaza-s-indacene-3-styryloxyacetate and a 4,4-difluoro-5-phenyl-4-bora-3a,4a-diaza-s-indacene-3-styryloxyacetate. These molecules provided an unexpected and substantial increase in the signal intensity observed over prior art dyes (see, FIG. 5). The brightness of the new red BODIPY dye is superior over the prior red dyes.

Also provided by the present invention as a composition of matter, is a 4,4-difluoro-5-(4-methoxyphenyl)-4-bora-3a,4a-diaza-s-indacene-3-propionic acid is provided by the present invention. The composition is particularly useful in applications that require increased signal to noise levels and sharp spectral resolution, as in applications that employ more than one fluorophore.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 illustrates chemical structures of BODIPYs B410, B411 and 542/563 that are covalently attached to an oligonucleotide, indicated as R931.

FIG. 2 shows the effect of adding a styryloxy component to a blue BODIPY fluorophore 503/512 to yield BODIPY 410.

FIG. 3 shows the effect of adding styryloxy component to a green BODIPY fluorophore 523/547 to yield BODIPY411.

FIG. 4 shows the excitation/emission spectra of the new yellow BODIPY fluorophore, B542/563.

FIG. 5 shows the intensity of BODIPY fluorophore B410 after excitation at 514 nm as compared to BOPIPY 567/589, BODIPY 581/591, and BODIPY 589/600.

FIG. 6 shows the spectral resolution of the new set of eight resolvable BODIPY fluorophores, including the three new BODIPY dyes.

FIG. 7 shows chemical modifications of BOPIPY fluorphores.

FIG. 8. shows the change in emission wavelengths (nm) that result after the modifications shown in FIG. 7.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, BET refers to a BODIPY energy transfer (BET) primer, such as those described in U.S. Pat. No. 5,614,386 to Metzker et al. The BET is a double-labeled primer having a donor or an acceptor and is labeled at the 3′-end position for labeling nucleic acids, including ribonucleotides, deoxyribonucleotides and dideoxrinucleotides for 8-color sequencing. For example, a BET primer is first labeled internally with a first BODIPY at a first site, and this internal label is the acceptor or the donor. Subsequent labeling at a second site, which was protected during the first labeling reaction and is deprotected prior to the adding of a second BODIPY dye, at the 3′-end position with a second BODIPY produces the donor, if the internal label is the acceptor, or the acceptor, if the internal label is the donor.

As used herein, “acceptor” refers to a fluorophore that functions as a quencher fluorophore when in close proximity to an donor fluorophore. The oligonucleotides of the present invention having an acceptor also have a donor, which improves signal intensity. The acceptor typically has a maximum excitation and fluorescence. The attachment of the acceptor is at a position most 5′ on the labeled oligonucleotide, a position most 3′ on the labeled oligonucleotide or internally on the labeled oligonucleotide, provided that the acceptor is attached at a position different from the donor.

As used herein, “BODIPY” shall refer to a broad class of modified, spectrally-discriminating fluorophores wherein the parent heterocyclic molecule is a dipyrrometheneboron difluoride compound. Specific BODIPY fluorophores useful in the present invention include BODIPYs with adsorption maxima of about 450 to about 700, and emission maxima of about 450 to about 700. Preferred embodiments include BODIPYs with adsorption maxima of about 500 to about 700 nm, and emission maxima of about 500 to about 700 nm. Examples of preferred embodiment BODIPYs include BODIPY 503/512 (4,4-difluoro-5,7-dimethyl-4-bora-3a,4a-diaza-s-indacene-3-propionic acid), BODIPY 523/547 (4,4-difluoro-5-phenyl-4-bora-3a,4a-diaza-s-indacene-3-propionic acid), BODIPY 542/563 (4,4-difluoro-5-(4-methoxyphenyl)-4-bora-3a,4a-diaza-s-indacene-3-propionic acid), BODIPY B410 (4,4-difluoro-5,7-dimethyl-4-bora-3a,4a-diaza-s-indacene-3-styryloxyacetate), BODIPY 581/591 (4,4-difluoro-5-(4-phenyl-1,3-butadienyl)-4-bora-3a,4a-diaza-s-indacene-3-propionic acid), BODIPY B411 (4,4-difluoro-5-phenyl-4-bora-3a,4a-diaza-s-indacene-3-styryloxyacetate), BODIPY 630/650 (4,4-difluoro-3,5-bis-(2-thienyl)-4-bora-3a,4a-diaza-s-indacene-3-styryloxyacetate), and BODIPY 650/665 (4,4-difluoro-5-(2-pyrrolyl)-4-bora-3a,4a-diaza-s-indacene-3-styryloxyacetate). The BODIPY fluorophores of the present invention have a linker at the 3 position of the BODIPY molecule that has at least one functional group capable of attachment to a 5 position of a pyrimidine or a 7 position of a purine and the 5′-end, internal, or 3′-end position of an oligonucleotide.

The BODIPY 542/563 molecule is that which is defined herein to have a propionic acid linker and unexpectedly provided an enhanced absolute intensity and improved spectral resolution over the prior art molecule.

As used herein a “chemically modified” BODIPY has been chemically modified so that the BODIPY fluorophore is used to replace a prior art 5′-end labeled fluorophore in polynucleotide sequencing and conventional software in used.

As used herein, the term “DNA sequencing” refers to the process or method of determining the nucleic acid sequence of a polynucleotide.

As used herein, the “donor” refers to a fluorophore that functions as a quenched fluorophore when in close proximity to an acceptor. The donor fluorophore is attached to the oligonucleotide at a position most 3′ on the labeled oligonucleotide, at a position most 5′ on the labeled oligonucleotide, or at a position internal on the labeled oligonucleotide, provided that the donor is attached at a position different from the acceptor.

As used herein, the term “linker” or “linker arm” refers to a molecule that tethers or “links” a dye to a primer, a ribonucleotide, a deoxyribonucleotide, or a dideoxyribonucleotide. Typical linker molecules include alkanes of various lengths.

As used herein, “labeled oligonucleotide” refers to the oligonucleotide in the sequencing assay that is labeled with at least one BODIPY fluorophores.

As used herein, electrophoresis “lanes”, “tracks”, “columns” or “capillary” refers to the particular path in the electrophoretic medium in which the sequencing products are run and detected. For example, the sequencing products terminating in dideoxyadenosine triphosphate, dideoxycytidine triphosphate, dideoxyguanosine triphosphate or dideoxythymidine triphosphate are run in four, five, six, seven or eight lanes, or if differentially labeled, are run in the same lane.

As used herein, “5′-end position” refers to the 5′-end position on the deoxyribose moiety of a polynucleotide.

As used herein, “3′-end position” refers to the 3′-end position on the deoxyribose moiety of a nucleotide.

As used herein, “corresponding” means identical to or complementary to a designated nucleotide sequence.

If two different, non-overlapping oligonucleotides anneal to different regions of the same linear complementary nucleic acid sequence, the 3′ end of one oligonucleotide points toward the 5′ end of the other; the former is called the “upstream” oligonucleotide and the latter the “downstream” oligonucleotide.

The complement of a nucleic acid sequence as used herein refers to an oligonucleotide which, when aligned with the nucleic acid sequence such that the 5′ end of one sequence is paired with the 3′ end of the other, is in antiparallel association. Certain bases not commonly found in natural nucleic acids may be included in the nucleic acids of the present invention include, for example, inosine and 7-deazaguanine. Complementarity need not refer to entirely matched; stable duplexes may contain mismatched base pairs or unmatched bases. Those skilled in the art of nucleic acid technology recognize that duplex stability is determined empirically by considering a number of variables including, for example, the length of the oligonucleotide, percent concentration of cytosine and guanine bases in the oligonucleotide, ionic strength, and incidence of mismatched base pairs.

As used herein, “fluorescence” is defined as the emission of light by a substance when it is stimulated by light. This phenomenon occurs when the application of a stimulus (light) causes electrons contained in the specimen to enter higher energy states (excited states). When these electrons revert to their original energy state (ground state), the excess energy is released in the form of light. A substance must absorb light to emit fluorescence. The wavelength of emission is generally longer than the wavelength of the excitation light. The intensity of the fluorescence is proportional to the intensity of the excitation light. Each substance possesses a characteristic fluorescence spectrum.

An illuminating beam emits light through an excitation filter, which transmits only the specific wavelength necessary to induce fluorescence. An optical filter is used to separate excitation light from emission light to make the object studied visible. The difference in wavelength between the apex, or maxima, of the absorption and emission spectra of a fluorophore is referred to as the Stokes shift, or Red Shift.

As used herein, “fluorophores” comprise the following characteristics. Fluorophore conjugation, or linking, to the molecule of choice must be relatively easy. The fluorophore must give a strong fluorescence and resist fading over time. The absorption and emission maxima of a fluorphore must be reasonably far apart. Fluorophores are said to be “spectrally resolved” in relation to one another if their emission spectra allow individual identification of each fluorophore.

The term “label” as used herein refers to any atom or molecule which is used to provide a detectable (preferably quantifiable) signal, and which is attached to a nucleic acid or protein. Labels provide signals detectable by fluorescence spectroscopy, radioactivity, colorimetry, X-ray diffraction or absorption, magnetism, enzymatic activity, and the like, but preferably′ by fluorescence spectroscopy.

As used herein, “5′→3′ nuclease activity” or “5′ to 3′ nuclease activity” refers to that activity of a template-specific nucleic acid polymerase including either a 5′→3′ exonuclease activity traditionally associated with some DNA polymerases whereby nucleotides are removed from the 5′ end of an oligonucleotide in a sequential manner, (i.e., E. coli DNA polymerase I has this activity whereas the Klenow fragment does not), or a 5′ to 3′ endonuclease activity wherein cleavage occurs more than one nucleotide from the 5′ end, or both.

The term “primer,” as used herein, is meant to encompass any nucleic acid that is capable of priming the synthesis of a nascent nucleic acid in a template-dependent process. In specific embodiments, the primers are oligonucleotides of about ten base pairs in length. In other specific embodiments, the primers are about twenty or thirty base pairs in length, and longer sequences are also contemplated.

As used herein, “Taqman®” or “Taqman® assay” refers to assays that utilize the 5′ to 3′ exonuclease activity of Taq polymerase in a polymerase chain reaction to generate a specific detectable signal concomitantly with amplification. An oligonucleotide probe, nonextendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. The assay is sensitive and specific and is a significant improvement over more cumbersome detection methods. In one such assay, the oligonucleotide that is degraded has at least two light-emitting fluorophores attached. The fluorophores interact each other to modify (quench) the light emission of the fluorophores. The 5′-most fluorophore is the quencher fluorophore. The 3′-most fluorophore is the quenched fluorophore. In another type of Taqman® assay, an oligonucleotide probe is labeled with a light-emitting quenched fluorophore wherein the reaction is carried out in the presence of a DNA binding compound (quenching agent) that interacts with the fluorophore to modify the light emission of the label. A labeled oligonucleotide in the Taqman® assay is labeled with at least two BODIPY fluorophores.

As used herein, “quenched” refers to the interaction of the at least two BODIPY fluorophores, referred to as an acceptor and a donor, on the labeled oligonucleotide. Both BODIPY fluorophores are present on the labeled oligonucleotide and fluorescence of either fluorophore is not detected.

As used herein, “quencher agent” refers to intercalating compounds and the like similar to ethidium bromide for use in a Taqman® assay similar to that used in the method of Fisher, et al., U.S. Pat. No. 5,491,063.

The term “nucleic acid” generally refers to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 10 nucleobases in length. As used herein, the term “polynucleotide” overlaps with the term “oligonucleotide”, and the polynucleotides detected in the methods of the present invention include a molecule of about 18 nucleobases and larger, wherein the polynucleotides are extended primer products by means of a DNA polymerase and comprise fluorescent labels that are detected by automated DNA sequencing instrumentation.

These definitions generally refer to at least one single-stranded molecule, but in specific embodiments also encompass at least one additional strand that is partially, substantially or fully complementary to the at least one single-stranded molecule. Thus, a nucleic acid encompasses at least one double-stranded molecule or at least one triple-stranded molecule that comprises one or more complementary strand(s) or “complement(s)” of a particular sequence comprising a strand of the molecule. As used herein, a single stranded nucleic acid is denoted by the prefix “ss” and a double stranded nucleic acid by the prefix “ds”.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions, which tolerate little, if any, mismatch between a nucleic acid and a target strand.

As used herein, “hybridization” or “hybridizes” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions comprise, for example, low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

As used herein, “denaturation” is defined as the breaking of hydrogen bonds between the two strands of DNA and the separating of double-stranded DNA into two single stranded molecules. Denaturation may occur under several conditions. Salt is needed to keep DNA in a double helix, as positively charged cations will neutralize the negatively charged phosphate groups on the DNA molecule. Thus, DNA will denature in distilled water containing no salts. Hydrophobic solvents will disrupt interactions between the hydrophobic bases, thus denaturing DNA. Increased temperature will break hydrogen bonds and cause denaturation of DNA. Alkali base will change the polarity of groups involved in hydrogen bonds. Above pH 11.3 hydrogen bonds are disrupted and DNA is denatured. In a preferred embodiment, DNA is denatured through′ increased temperature.

A skilled artisan is aware that a nucleic acid is made by any technique known to in the art. Non-limiting examples of synthetic nucleic acid, particularly a synthetic oligonucleotide, include a nucleic acid made by in vitro chemically syntheses using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. Ser. No. 5,705,629, each incorporated herein by reference. A non-limiting example of enzymatically produced nucleic acid include one produced by enzymes in amplification reactions such as PCRTM (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A′ non-limiting example of a biologically produced nucleic acid includes recombinant nucleic acid production in living cells, such as recombinant DNA vector production in bacteria (see for example, Sambrook et al 1989, incorporated herein by reference).

Generally, a nucleic acid to be subject to a sequencing assay requires purification. A nucleic acid may be purified on polyacrylamide gels, cesium chloride centrifugation gradients, or by any other means known to one of ordinary skill in the art (see for example, Sambrook et al. 1989, incorporated herein by reference).

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

One or more nucleic acid(s) may comprise, or be composed entirely of, at least one derivative or mimic of at least one nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refers to a molecule that may or may not structurally resemble a naturally occurring molecule, but functions similarly to the naturally occurring molecule. As used herein, a “moiety” generally refers to a smaller chemical or molecular component of a larger chemical or molecular structure, and is encompassed by the term “molecule.”

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U). Non-limiting examples of derivatives or mimic of ourines and pyrimidines are given in Table 1.

Nucleobase, nucleoside and nucleotide mimics or derivatives are well known in the art, and have been described in exemplary references such as, for example, Scheit, Nucleotide Analogs (John Wiley, New York, 1980), incorporated herein by reference. “Purine” and “pyrimidine” nucleobases encompass naturally occurring purine and pyrimidine nucleobases and also derivatives and mimics thereof, including but not limited to, those purines and pyrimidines substituted by one or more of alkyl, caboxyalkyl, amino, hydroxyl, halogen (i.e. fluoro, chloro, bromo, or iodo), thiol, or alkylthiol wherein the alkyl group comprises of from about 1, about 2, about 3, about 4, about 5, to about 6 carbon atoms. Non-limiting examples of purines and pyrimidines include deazapurines, 2,6-diaminopurine, 5-fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcyosine, 5-bromouracil, 5-ethyluracil, 5-iodouracil, 5-chlorouracil, 5-propyluracil, thiouracil, 2-methyladenine, methylthioadenine, N,N-diemethyladenine, azaadenines, 8-bromoadenine, 8-hydroxyadenine, 6-hydroxyaminopurine, 6-thiopurine, 4-(6-aminohexyl/cytosine), and the like. In specific embodiments of the present invention, a purine and/or pyrmidine derivative or mimic is employed to, for example, label an olibonucleotide, such as a sequence primer. A table of exemplary, but not limiting, purine and pyrimidine derivatives and mimics is also provided herein below.

TABLE 1
Purine and Pyrmidine Derivatives or Mimics

| Abbr. | Modified base description | Abbr. | Modified base description |
|---|---|---|---|
| ac4c | 4-acetyleytidine | mam5s2u | 5-methoxyaminomethyl-2- |
| | | | thiouridine |
| chm5u | 5- | man q | Beta,D-mannosylqueosine |
| | (carboxyhydroxylmethyl)uridine | | |
| Cm | 2′-O-methylcytidine | mcm5s2u | 5-methoxycarbonylmethyl-2- |
| | | | thiouridine |
| cmnm5s2u | 5-carboxymethylaminomethyl- | mcm5u | 5-methoxycarbonylmethyluridine |
| | 2-thioridine | | |
| cmnm5u | 5- | mo5u | 5-methoxyuridine |
| | carboxymethylaminomethyluridine | | |
| D | Dihydrouridine | ms2i6a | 2-methylthio-N6- |
| | | | isopentenyladenosine |
| Fm | 2′-O-methylpseudouridine | ms2t6a | N-((9-beta-D-ribofuranosyl-2- |
| | | | methylthiopurine-6- |
| | | | yl)carbamoyl)threonine |
| gal q | beta,D-galactosylqueosine | mt6a | N-((9-beta-D-ribofuranosylpurine-6- |
| | | | yl)N-methyl-carbamoyl)threonine |
| Gm | 2′-O-methylguanosine | mv | Uridine-5-oxyacetic acid |
| | | | methylester |
| I | Inosine | o5u | Uridine-5-oxyacetic acid (v) |
| i6a | N6-isopentenyladenosine | osyw | Wybutoxosine |
| m1a | 1-methyladenosine | p | Pseudouridine |
| m1f | 1-methylpseudouridine | q | Queosine |
| m1g | 1-methylguanosine | s2c | 2-thiocytidine |
| mlI | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | t | 5-methyluridine |
| m3c | 3-methylcytidine | t6a | N-((9-beta-D-ribofuranosylpurine-6- |
| | | | yl)carbamoyl)threonine |
| m5c | 5-methylcytidine | tm | 2′-O-methyl-5-methyluridine |
| m6a | N6-methyladenosine | urn | 2′-O-methyluridine |
| m7g | 7-methylguanosine | yw | Wybutosine |
| mam5u | 5-methylaminomethyluridine | x | 3-(3-amino-3- |
| | | | carboxypropyl)uridine, (acp3)u |

As used herein, “nucleoside” refers to an individual chemical unit comprising a nucleobase covalently attached to a nucleobase linker moiety. A non-limiting example of a “nucleobase linker moiety” is a sugar comprising 5-carbon atoms (a “5-carbon sugar”), including but not limited to deoxyribose, ribose or arabinose, and derivatives or mimics of 5-carbon sugars. Non-limiting examples of derivatives or mimics of 5-carbon sugars include 2′-fluoro-2′-deoxyribose or carbocyclic sugars where a carbon is substituted for the oxygen atom in the sugar ring. By way of non-limiting example, nucleosides comprising purine (i.e. A and G) or 7-deazapurine nucleobases typically covalently attach the 9 position of the purine or 7-deazapurine to the 1′-position of a 5-carbon sugar. In another non-limiting example, nucleosides comprising pyrimidine nucleobases (i.e. C, T or U) typically covalently attach the 1 position of the pyrimidine to 1′-position of a 5-carbon sugar (Kornberg and Baker, DNA Replication, 2nd Ed. (Freeman, San Francisco, 1992). However, other types of covalent attachments of a nucleobase to a nucleobase linker moiety are known in the art, and non-limiting examples are described herein.

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

U.S. application Ser. No. 07/943,516, filed Sep. 11, 1992, and its corresponding published PCT application WO 94/06815, describe other novel amine-containing compounds and their incorporation into oligonucleotides for, inter alia, the purposes of enhancing cellular uptake, increasing lipophilicity, causing greater cellular retention and increasing the distribution of the compound within the cell.

Additional non-limiting examples of nucleosides, nucleotides or nucleic acids comprising 5-carbon sugar and/or backbone moiety derivatives or mimics are provided in Table 2 herein below.

TABLE 2
Nucleic Acid Derivatives or Mimics

| Type of Modification | Properties | U.S. Pat. No. |
|---|---|---|
| Oligonucleotides comprising | Formation of triple helixes with target dsDNA to | 5,665,541 |
| purine base modified as the 8 | detect and/or prevent function or expression of | |
| position such as 8-oxo-adenine or | dsDNA. | |
| 8-oxo-guanine. | | |
| A nucleoside analog is used in | Enhanced versatility in hybridization | 5,681,947 |
| degenerative positions in the | applications | |
| oligonucleotide sequence. | | |
| Nucleic acids incorporating | Fluorescent nucleic acids probes. | 5,652,099, |
| fluorescent analogs of | | 5,763,167 |
| nucleosides found in DNA or | | |
| RNA. | | |
| Oligonucleotides analogs with | Enhance nuclease stability. | 5,614,617 |
| substitutions on pyrimidine rings. | | |
| Oligonucleotides and analogs | Ability of oligonucleotides to detect and | 5,670,663, |
| with modified 5-carbon sugars | modulate target gene expression. | 5872,232, |
| (i.e. modified 2′-deoxyfuranosyl | | 5,859,221 |
| moieties) | | |
| Oligonucleotide comprising at | Useful in hybridization assays and as therapeutic | 5,446,137 |
| least one 5-carbon sugar moiety | agents. | |
| substituted at the 4′ position with | | |
| a substituent other than hydrogen. | | |
| Oligonucleotides with both | Reduced immune stimulation, and reduced | 5,886,165 |
| deoxyribonucleotides with 3′-5′ | complement and coagulation effects. | |
| internucleotide linkages and | | |
| ribonucleotideswith 2′-5′ | | |
| internucleotide linkages. | | |
| A modified internucleotide | Enhanced nuclease resistance | 5,714,606 |
| linkage wherein a 3′-position | | |
| oxygen of the internucleotide | | |
| linkage is replaced by a carbon. | | |
| Oligonucleotides containing one | Enhanced nuclease resistance | 5.672,697 |
| or more 5′ methylene | | |
| phosphonate internucleotide | | |
| linkages. | | |
| The linkage of a substituent | Oligonucleotides with enhanced nuclease | 5,466,786, |
| moiety which may comprise a | stability and ability to deliver drugs or detection | 5,792,847 |
| drug or label to the 2′ carbon of | moieties. | |
| an oligonucleotide. | | |
| Oligonucleotide analogs with a 2 | Enhanced cellular uptake, resistance to nucleases | 5,223,618 |
| or 3 carbon backbone linkage | and good hybridization to target RNA. | |
| attaching the 4′ position and 3′ | | |
| position of adjacent 5-carbon | | |
| sugar moiety. | | |
| Oligonucleotides comprising at | Useful as nucleic acid hybridization probe. | 5,470,967 |
| least one sulfamate or sulfamide | | |
| internucleotide linkage. | | |
| Oligonucleotides with three or | Improved nuclease resistance and cellular | 5,378,825, |
| four atom linker moiety replacing | uptake, useful in regulating RNA expression. | 5,777,092, |
| phosphodiester backbone moiety. | | 5,623,070, |
| | | 6,610,289, |
| | | 5,602,240 |
| Hydrophobic carrier agent | Enhanced membrane permeability and stability | 5,858,988 |
| attached to the 2′-O position of | | |
| oligonucleotides. | | |
| Olignucleotides conjugated to | Enhanced hybridization to DNA or RNA; | 5,214,136 |
| anthraquinone at the 5′ terminus | enhanced stability to nucleases. | |
| PNA-DNA-PNA chimeras | Enhanced nuclease resistance, binding affinity, | 5,700,922 |
| wherein the DNA comprises 2′- | and ability to activate RNase H. | |
| deoxy-erythro-pentofuranosyl | | |
| nucleotides. | | |
| RNA linked to a DNA to form a | | 5,708,154 |
| DNA-RNA hybrid | | |

The practice of the present invention employs, unless otherwise indicated, conventional techniques of molecular biology, microbiology and recombinant DNA techniques, which are within the skill of the art. Such techniques are explained fully in the literature. See, e.g., Sambrook, Frtisch & Maniatis, Molecular Cloning; A Laboratory Manual, Second Edition (1989); Oligonucleotide Synthesis (M. J. Gait, ed., 1984); Nucleic Acid Hybridization (B. D Hames & S. J. Higgins, eds., 1984); A Practical Guide to Molecular Cloning (B. Perbal, 1984); and a series, Methods in Enzymology (Academic Press, Inc.). each incorporated by reference herein.

C. The Present Invention

DNA sequencing assays predominantly employ a 4-color sequencing assay and are relegated to sequencing a single-strand of DNA, such as a sense strand, with four spectrally differentiated dyes in a first reaction followed by a second reaction using the same 4-color dyes to obtain sequence information of the complementary strand, such as in this case the antisense strand. The invention described herein provides a method to detect up to eight oligonucleotides, ribonucleotides, deoxynucleotides, or dideoxyribonucleotides, that are differentially-labeled with a fluorophore, wherein the fluorophore comprises a substituted 4,4-difluoro-4-bora-3A,4A-diaza-s-indacene (BODIPY fluorophore) compound. Each ogligonucleotide, ribonucleotide, deoxynucleotide or dideoxyribonucleotide is labeled with a different fluorophore as defined by the absorption/emission maxima of the fluorophore, thus, up to eight of the labeled nucleotides are detected simultaneously. Determining the sequence of the sense and antisense strands involves identifying specific nucleotides at each position.

It is known in the art that BODIPY fluorophores have improved spectral characteristics, narrower band width, insensitivity to solvent or pH, and improved photostability compared to conventional fluorescein and rhodamine dyes. U.S. Pat. Nos. 5,614,386, 5,861,287 and 5,994,063 as incorporated by reference in their entirety. As described herein, the new substituted 4,4-difluoro-4-bora-3A,4A-diaza-s-indacenes (BODIPY fluorophores) provide a bathochromic shift as compared to previously described BODIPY fluorophores and an unexpected improvement in the spectral resolution such that a set of eight spectrally resolvable compounds useful for 8-color sequencing of a polynucleotide, for simultaneous detection of forward and reverse DNA sequencing reactions, for preparation of BETS, for homogeneous assays such as Taqman®, for hybridization of nucleic acids, and any method that benefits from having high spectal resolution together with high sample throughput are provided. The numerical designations of the reactions, polynucleotides, oligonucleotides, classes or the like as used herein are not limiting to the scope of the present invention. For example, a sixth class having any dideoxy analog is contemplated, or a first class having any deoxy analog is also contemplated, provided that each of the four nucleotides, deoxy, dideoxy or otherwise, are present for each strand in the step.

In 8-color sequencing reactions, eight sequencing reactions are generated from primed synthesis, which is carried out in the presence of enough of the dideoxy analog of one of four possible deoxynucleotides so that the growing chains are randomly terminated by the incorporation of 2′, 3′-dideoxynucleotides using DNA polymerase. The reaction also includes the natural 2′-deoxynucleotides, which extend the DNA chain by DNA synthesis. Thus, balanced appropriately, competition between chain extension and chain termination results in the generation of a set of nested DNA fragments, which are uniformly distributed over thousands of bases and differ in size as base pair increments. In specific embodiments, the sequencing primer is labeled with a characteristic fluorophore, meaning a fluorophore having a distinct and spectrally resolved fluorescence as compared to another fluorophore used to label another primer. In other specific embodiments, the dideoxy analog is labeled with a characteristic fluorophore.

The fluorophore may be at least one BODIPY fluorophore which has been chemically modified so that the BODIPY fluorophore is used to replace a prior art 5′-end labeled fluorophore in polynucleotide sequencing and conventional software in used. The BODIPY fluorophore is used in one out of eight reactions, two out of eight reactions, three out of eight reactions, four out of eight reactions, five out of eight reactions, six out of eight reactions, seven out of eight reactions, and eight out of eight reactions. In a specific embodiment, the first set of fluorophores comprises at least one BODIPY fluorophore selected from the group consisting of BODIPY 542/563, BODIPY B410, BODIPY B411, BODIPY 503/512, BODIPY 523/547, BODIPY 581/591, BODIPY 630/650, and BODIPY 650/665. The fluorophores alter the mobility of the corresponding termination products fin the same way, thereby nullifying the need for software correction to generate evenly-spaced ribonucleic acid, deoxyribonucleic acid and dideoxyribonucleic acid sequences.

The set of fluorophores may further comprise fluoresceins, rhodamines, cyanines, coumarins, sulfonated pyrenes, squaraines or alexas. It is contemplated that a fluorophore that provides a spectrally resolved absorption/emission maxima and, preferably, a high signal intensity, is useful as a further embodiment to the present invention.

The first set of fluorophores may comprise BODIPY 542/563 or BODIPY B410 or BODIPY B411. In a further specific embodiments, the set may further comprise fluoresceins, rhodamines, cyanines, coumarins, sulfonated pyrenes, squaraines or alexas, and in yet a further specific embodiment, the set of fluorophores further comprises BODIPY 503/512, BODIPY 523/547, BODIPY 581/591, BODIPY 630/650, and BODIPY 650/665. The BODIPY 542/563 molecule is that which is defined herein to have a propionic acid linker and unexpectedly provided an enhanced absolute intensity and improved spectral resolution over the prior art molecule.

It is also contemplated that the present invention provides a method for genetic analysis of DNA fragments wherein said DNA fragments are labeled with at least one BODIPY fluorophore selected from the group consisting of BODIPY B410, BODIPY B411, and BODIPY 542/563.

The second set of fluorophores may comprise at least one BODIPY fluorophore selected from the group consisting of BODIPY 542/563, BODIPY B410, BODIPY B411, BODIPY 503/512, BODIPY 523/547, BODIPY 581/591, BODIPY 630/650, and BODIPY 650/665. In further specific embodiments, the second set of fluorophores further comprises fluoresceins, rhodamines, cyanines, coumarins, sulfonated pyrenes, squaraines or alexas.

The second set of fluorophores may also comprise BODIPY 542/563 or BODIPY B410, or BODIPY B411. In a further specific embodiments, the set further comprises fluoresceins, rhodamines, cyanines, coumarins, sulfonated pyrenes, squaraines or alexas, and in yet a further specific embodiment, the set of fluorophores further comprises BODIPY 503/512, BODIPY 523/547, BODIPY 581/591, BODIPY 630/650, and BODIPY 650/665.

Each fluorophore in the first set and the second set may exhibit a characteristic adsorption maxima that is spectrally resolved as compared to the other fluorophores employed, and each fluorophore has an adsorption maxima in the range of about 500 to about 700.

The step of electrophoretically separating the polynucleotides in the first mixture and the second mixture is performed on the same gel or on different gels. Further, the different classes of polynucleotides within the mixtures are electrophoresed on the same gel or on separate gels. Thus, the fluorophores in the first set and the second set are different with respect to absorption/emission maxima, and as well, the fluorophores comprising each set are characteristic and specific from one another.

The designation of, for example, the eighth class having a terminal dideoxythymidine is not meant to be limiting the scope of the eight reactions, in that a sixth class of polynucleotides have a terminal dideoxythymidine provided that the eighth class has a terminal dideoxycytidine. Thus, as long as the four dideoxy analogs are present, the exact class designations are not limiting.

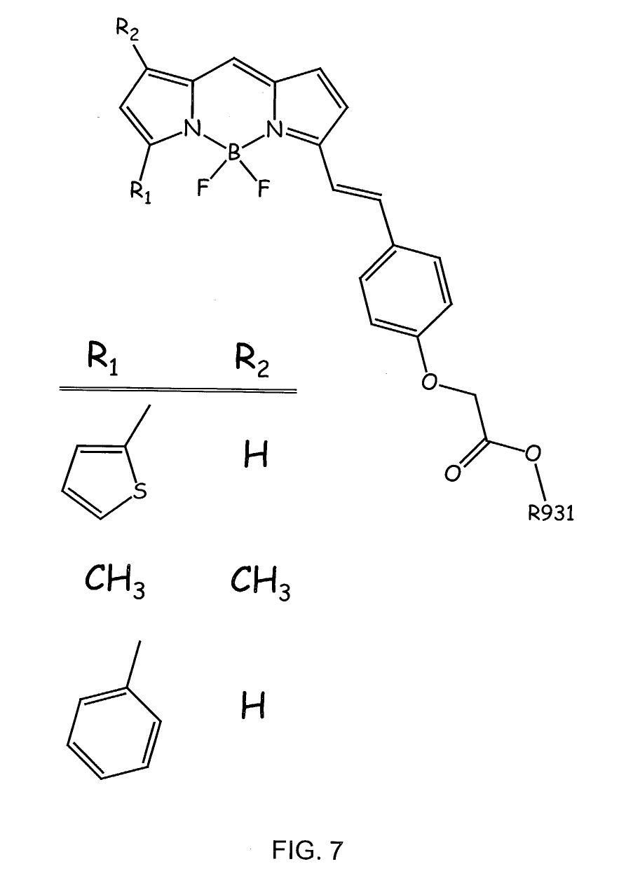

The present invention provides a set of substituted 4,4-difluoro-4-bora-3A,4A-diaza-s-indacene (BODIPY fluorophore) compounds suitable for performing an 8-color polynucleotide sequencing assay. Due to the improved spectral characteristics demonstrated in the set of dyes described herein (see, FIG. 7), the use of BODIPY fluorophores leads to improved polynucleotide, in particular DNA, sequencing. Further, because of the lack of an effect (or lack of a differential effect) on electrophoretic mobility, their use leads to improved automated DNA sequencing. Additionally, the distinct spectral characteristics of the compounds of the present invention are such that an 8-color DNA sequence assay is performed therewith. Thus, the improved spectral resolution of the compounds described herein allow concomitant sequencing of a single-strand of a polynucleotide (sense strand) and a complementary strand of the same polynucleotide (antisense strand).

Thus, the present invention describes a set of fluorophores and methods suitable for 8-color sequencing of polynucleotides, thereby overcoming problems in the prior art including compromising in sensitivity of a sequencing detection system, increasing cost of providing separate excitation sources for each dye, and changes in electrophoretic mobility resulting in extensive band broadening or reversal of band positions on the gel.

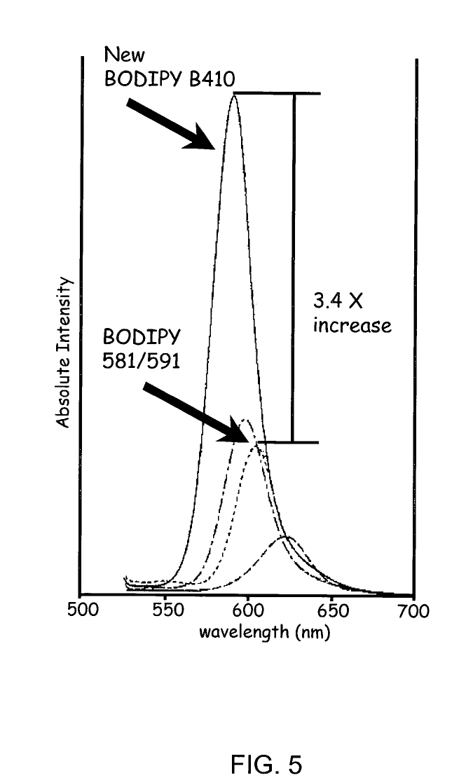

As has been touched upon, spectral resolution of the dye components is beneficial and preferred in the application of the various methods of the present invention. To that end, the present invention also provides as compositions of matter, a 4,4-difluoro-5,7-dimethyl-4-bora-3a,4a-diaza-s-indacene-3-styryloxyacetate and a 4,4-difluoro-5-phenyl-4-bora-3a,4a-diaza-s-indacene-3-styryloxyacetate. The molecules provided an unexpected and substantial increase in the signal intensity observed over prior art dyes (see, FIG. 5). The brightness of the new red BODIPY dye is superior over the prior red dyes.

Also provided by the present invention as a composition of matter, is a 4,4-difluoro-5-(4-methoxyphenyl)-4-bora-3a,4a-diaza-s-indacene-3-propionic acid is provided by the present invention. The composition is particularly useful in applications that require increased signal to noise levels and sharp spectral resolution, as in applications that employ more than one fluorophore.

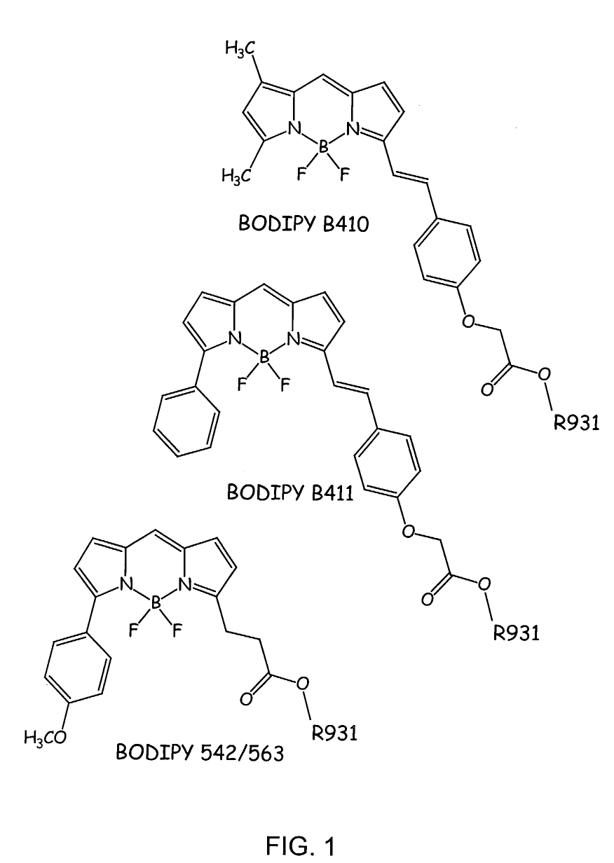

A class of dyes, 4,4-difluoro-4-bora-3A,4A-diaza-s-indacene BODIPY fluorophores has been described. The parent heterocyclic molecule of the BODIPY fluorophore is a dipyrrometheneboron difluoride compound and which is modified to create a broad class of spectrally-discriminating fluorophore. In the present invention, there are three new novel BODIPY derivatives which exhibit an increased fluorescence quantum yield and a significant red shift relative to the BODIPY dyes of the prior art. The structures of the three new BODIPY dyes, bound to an oligonucleotide primer, are shown in FIG. 1. In BODIPY B410, methyl groups are introduced in the 5 and 7 position of the central dipyrrometheneboron difluoride moiety, along with the addition, at the 3 position of a styroxyl group, to which the oligonucleotide (indicated as R931) is bound. BODIPY B410 is 4,4-difluoro-5,7-dimethyl-4-bora-3a,4a-diaza-s-indacene-3-styryloxyacetate. BODIPY B411, 4,4-difluoro-5-phenyl-4-bora-3a,4a-diaza-s-indacene-3-styryloxyacetate, differs from BODIPY B410 in that the two methyl groups at the 5 and 7 positions are replaced by a phenyl and a proton, respectively. Finally, BODIPY 542/563, 4,4-difluoro-5-(4-methoxyphenyl)-4-bora-3a,4a-diaza-s-indacene-3-propionic acid, has a methoxyphenyl group at position 5 and a propionate group at position 3. The oligonucleotide is bound to the functional group at the 3 position by an ester linkage in all cases.

### EXAMPLES

The following examples are included to demonstrate aspects of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Effect of Varying R₁ and R₂

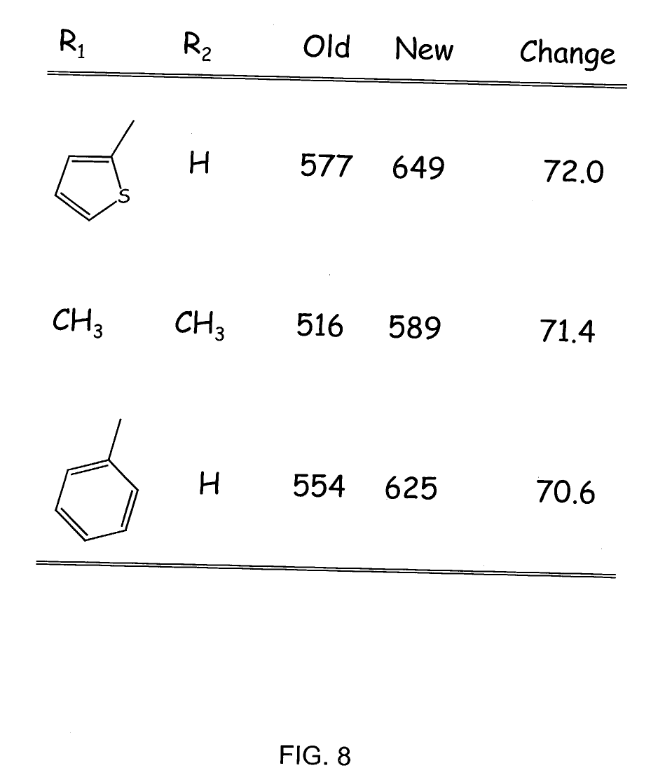

The 3-linker substitutions and their effect on the fluorescence spectrum of the BODIPY fluorophore were investigated. The structures analyzed are shown in FIG. 7, and the results are summarized in tabular form in FIG. 8. The substitution of slightly electron-withdrawing methyls (R₁ and R₂=CH₃) led to a 71.4 nanometer shift. A single substitution to the phenyl substituent or the thienyl substituent effected a 70.6 nanometer and 72.0 nanometer bathochromic shift, respectively. The data suggest that the styryloxy group generates a bathochromic shift for several BODIPY dyes in a reproducible manner.

### Example 2

### BODIPY Chemical Modification and Synthesis

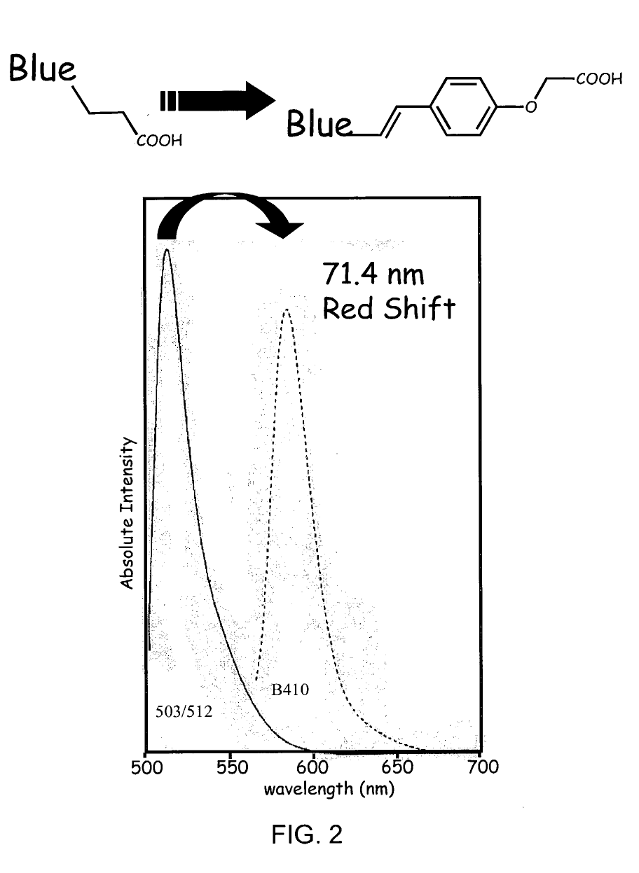

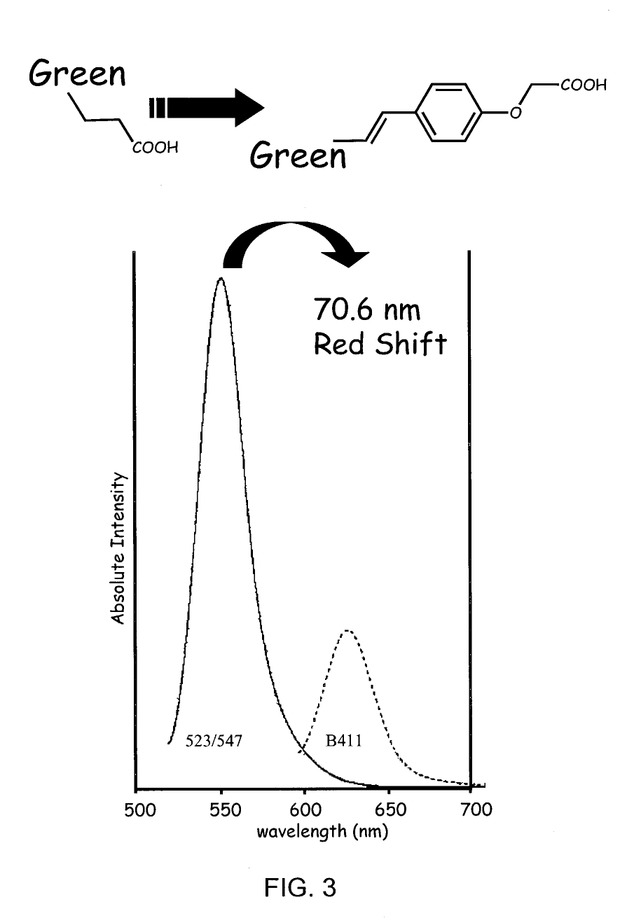

The styroxyl group introduces some useful and interesting photochemistry to the BODIPY core. FIG. 2 shows the emission spectra of BODIPY 503/512 (left, solid) and the new BODIPY B410 (right, dashed). A 71.4 nm, red shift is seen for BODIPY B410 relative to BODIPY 503/512. This shift to longer wavelengths is useful in that it is in a spectral region less likely to possess interfering emissions from other species. FIG. 3 demonstrates the effect of the styroxyl group on BODIPY 523/547; the resulting fluorophore is the new BODIPY B411. Another very significant red shift (70.6 nm) is seen as a result of this conversion.

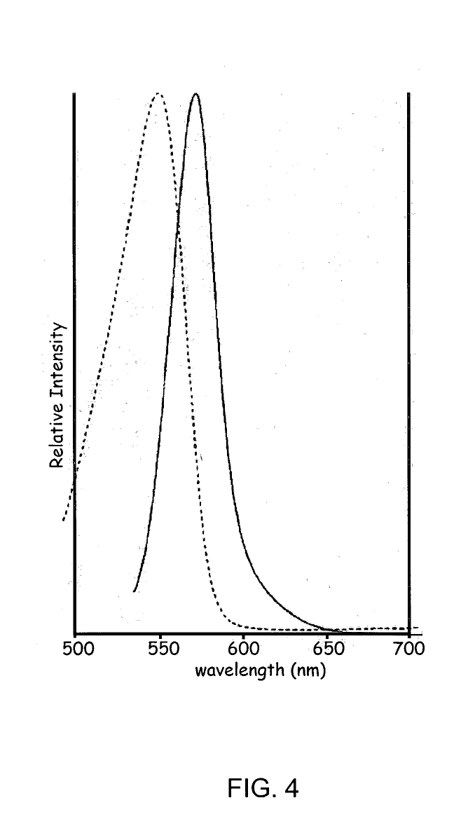

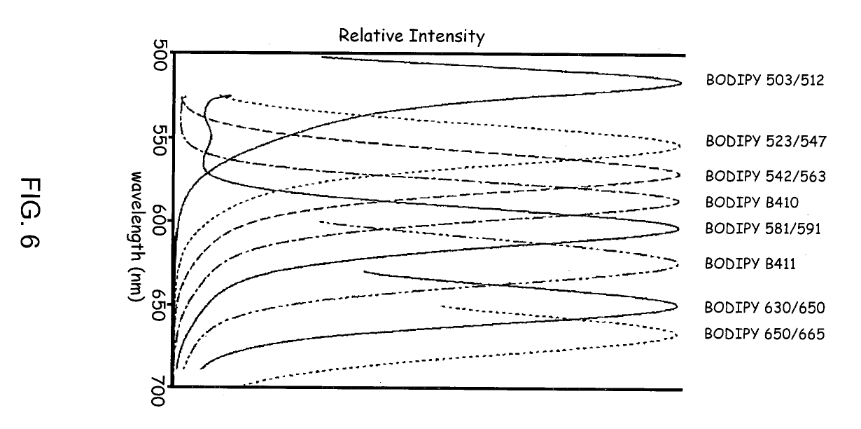

FIGS. 4 and 5 demonstrate the good quantum yields exhibited by two of the new BODIPY dyes. FIG. 4 demonstrates the excitation (right, dashed) and emission (left, solid) spectra of the new BODIPY 542/563. FIG. 5 shows the emission, at equal concentration of BODIPY B410, BODIPY 567/589, BODIPY 581/591, and BODIPY 589/600. All samples were excited at 514 nm and are at equal concentrations. As shown, BODIPY B410 exhibits a greater than 4-fold increase in fluorescence emission as compared to the prior art BODIPY 567/589. The emission spectra of the three new BODIPY dyes, along with five prior art BODIPY dyes is shown in FIG. 6. The emission of the new dyes fits nicely complement the prior art dyes, yielding eight spectrally resolved fluorescent dyes. These dyes are useful in the eight color sequencing method of the present invention.

One skilled in the art recognizes that it is possible to synthesize BODIPY 410, BODIPY 411, and BODIPY 542/563 using the general scheme for synthesis of BODIPY dyes. This consists of an acid catalyzed condensation of a 2-acylpyrrole or appropriately substituted 2-acylpyrrole with pyrrole or a substituted pyrrole having a hydrogen on the 2-position to give a dipyrromethene intermediate. Frequently there are two alternative routes whose choice depends primarily on the availability or ease of synthesis of the acyl pyrrole reactants. The dipyrromethene intermediate is condensed with borontrifluoride or a complex of boron trifluoride such as its etherate in the presence of a base to give the heterocyclic dye. Suitable bases include but are not limited to trimethylamine, triethylamine, tetramethylethylenediamine, and diazobicycloundecene. Suitable substitutents on the pyrroles include but are not limited to hydrogen, alkyl, cycloalkyl, aryl, arylalkyl and acyl. Dipyrrometheneboron difluoride products may be modified in a subsequent reaction by chemical techniques known to one skilled in the art including but not limited to sulfonation, nitration, alkylation, acylation, and halogenation. Furthermore, the substituents can in some cases be further modified to introduce chemically reactive functional groups that are understood to fall within the scope of this patent. Preferred side groups at R1 and R2 have been illustrated in FIG. 7. It is recognized that variations in the synthetic methods and reactants are possible that would fall within the scope and intent of this patent.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents

U.S. Pat. No. 5,994,063 to Metzker et al.

U.S. Pat. No. 5,861,287 to Metzker et al.

U.S. Pat. No. 5,728,529 to Metzker et al.

U.S. Pat. No. 5,614,386 to Metzker et al.

U.S. Pat. No. 6,087,099 to Gupte et al.

U.S. Pat. No. 4,318,846 to Khanna et al.

U.S. Pat. No. 4,811,218 to Hunkapiller et al.

U.S. Pat. No. 4,855,225 to Fung et al.

U.S. Pat. No. 5,366,860 to Bergot et al.

U.S. Pat. No. 5,188,934 to Menchen, et al.

U.S. Pat. No. 5,171,534 to Smith et al.

U.S. Pat. No. 5,241,060 to Englehardt, et al.

U.S. Pat. No. 4,755,458 to Rabbani, et al.

U.S. Pat. No. 5,151,507 to Hobbs, et al.

U.S. Pat. No. 5,171,534

U.S. Pat. No. 5,366,603

U.S. Pat. No. 5,378,825

U.S. Pat. No. 5,777,092

U.S. Pat. No. 5,623,070

U.S. Pat. No. 5,610,289,

U.S. Pat. No. 5,602,240

U.S. Pat. No. 5,665,541

U.S. Pat. No. 5,681,947

U.S. Pat. No. 5,652,099

U.S. Pat. No. 5,763,167

U.S. Pat. No. 5,614,617

U.S. Pat. No. 5,670,663

U.S. Pat. No. 5,872,232

U.S. Pat. No. 5,446,137

U.S. Pat. No. 5,886,165

U.S. Pat. No. 5,714,606

U.S. Pat. No. 5,859,221

U.S. Pat. No. 5,672,697

U.S. Pat. No. 5,466,786,

U.S. Pat. No. 5,792,847

U.S. Pat. No. 5,223,618

U.S. Pat. No. 5,470,967

U.S. Pat. No. 5,708,154

U.S. Pat. No. 5,700,922

U.S. Pat. No. 5,214,136

U.S. Pat. No. 5,858,988

U.S. Pat. No. 5,171,534

U.S. Pat. No. 5,210,015 to Gelfand et al.

U.S. Pat. No. 4,774,339

U.S. Pat. No. 5,491,063 to Fisher, et al

U.S. application Ser. No. 07/943,516, filed Sep. 11, 1992.

WO 94/06815

### Non-Patented Literature

Pringle et al., DNA Core Facilities Newsletter, 1:15-21 (1988)

Smith et al., Nature, 321:674-79 (1986).

Sambrook, Fritsch, Maniatis, In: Molecular Cloning: A Laboratory Manual, Vol. 1, Cold Spring Harbor Laboratory Press, Cold Spring Harbor, N.Y., Ch. 7,7.19-17.29, 1989.

Sanger et al., Proc. Natl. Acad. Sci., 74:5463-67 (1977)

Maxam et al., Proc. Natl. Acad. Sci., 74:560-564 (1977)

Drmanac et al., Genomics, 4:114-28 (1989)

Khrapko, FEB 256:118-22 (1989)

Sanger et al., J. Mol. Biol., 143:161-78 (1980)

Schreier et al., J. Mol. Biol., 129:169-72 (1979)

Smith et al., Nucleic Acids Research, 13:2399-2412 (1985)

Smith et al., Nature, 321:674-79 (1987)

Prober et al., Science, 238:336-41 (1987)

Section II, Meth. Enzymol., 155:51-334 (1987)

Church et al., Science, 240:185-88 (1988)

Swerdlow and Gesteland, Nucleic Acids Research, 18: 1415-19 (1989)

Ruiz-Martinez et al., Anal. Chem., 2851-58 (1993)

Studier, PNAS, 86:6917-21 (1989)

Kieleczawa et al., Science, 258:1787-91

Connell et al., Biotechniques, 5:342-348 (1987).

Haugland, et al., Molecular Probes: Handbook of Fluorescent Probes and Research Chemicals, pp. 24-32

Chen et al., J. Org. Chem., 65:2900-2906 (2000)

Metzker et al., Science 271:1420-1422 (1996)

Metzker et al., “Quantitation of mixed-base populations of HIV-1 variants by automated DNA sequencing with BODIPY dye-labeled primers” Biotechniques. 1998; 25(3):446-7, 450-2, 454.

Metzker et al., “Termination of DNA synthesis by novel 3′-modified-deoxyribonucleoside 5′-triphosphates” Nucleic Acids Res. 1994 22(20):4259-67.

Holland, et al., PNAS 88:7276-7280 (1991)

Muzny et al. “Using BODIPY dye-primer chemistry in large-scale sequencing” IEEE Eng Med Biol Mag. 1998; 17(6):88-93.

One skilled in the art readily appreciates that the present invention is well adapted to carry out the objectives and obtain the ends and advantages mentioned as well as those inherent therein. Sequencing, labeling, dyes, oligonucleotides, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A method of sequencing a sense strand and an antisense strand of a double-stranded polynucleotide comprising:
i) denaturing the double-stranded polynucleotide to provide the sense strand and the antisense strand;
ii) reacting the sense strand with a first set of four differentially labeled polynucleotides;
iii) reacting the antisense strand with a second set of four differentially labeled polynucleotides;
iv) identifying each of the eight polynucleotides by a fluorescence or an absorption spectrum of the fluorophore; and
v) determining the sequence of the sense strand from the polynucleotides differentially labeled with the first set of fluorophores and the sequence of the antisense strand from the polynucleotides differentially labeled with the second set of fluorophores.

2. The method of claim 1, wherein the fluorophore is at least one BODIPY fluorophore that has been chemically modified.

3. The method of claim 1, wherein said first set and second set of fluorophores are BODIPY 542/563, BODIPY B410, BODIPY B411, BODIPY 503/512, BODIPY 523/547, BODIPY 581/591, BODIPY 630/650, or BODIPY 650/665.

4. The method of claim 1, wherein said first set and second set of fluorophores are selected from the group consisting of fluoresceins, rhodamines, cyanines, coumarins, sulfonated pyrenes, squaraines and alexas.

5. The method of claim 1, wherein each fluorophore in the first set and the second set exhibits an adsorption maxima that is spectrally resolved as compared to the other fluorophores employed, and each fluorophore has an adsorption maxima in the range of about 500 to about 700 nm.

6. A method of 8-color sequencing of a polynucleotide comprising the steps of:
i) forming eight classes of polynucleotides wherein each class of polynucleotides is labeled with a fluorophore and each fluorophore is different;
ii) electrophoretically separating the classes of polynucleotides;
iii) illuminating the separated polynucleotides with a wavelength capable of causing the fluorophores to fluoresce; and
iv) identifying the classes of polynucleotides by the fluorescence or absorption spectrum of the fluorophores.

7. The method of claim 6, wherein the fluorophores comprise at least one BODIPY fluorophore selected from the group consisting of BODIPY 542/563, BODIPY B410, BODIPY B411, BODIPY 503/512, BODIPY 523/547, BODIPY 581/591, BODIPY 630/650 and BODIPY 650/665.

8. The method of claim 6, wherein the fluorophores are selected from the group consisting of fluoresceins, rhodamines, cyanines, coumarins, sulfonated pyrenes, squaraines and alexas.

9. The method of claim 6 wherein each of the fluorophores exhibits a characteristic adsorption maxima that is spectrally resolved as compared to the other fluorophores in the set, and the adsorption maxima of each of the fluorophores in the set is in the range of about 500 to about 700 nm.

10. The method of claim 6, wherein the electrophoretically separating the polynucleotides takes place in at least one lane of the gel.

11. The method of claim 6, wherein the eight fluorophores are linked to the 5′ ends of the polynucleotides.

12. The method of claim 6, wherein the eight fluorophores are linked to the 3′ ends of the polynucleotides.

13. A method of distinguishing polynucleotides having different 3′-terminal dideoxynucleotides in a chain termination method of DNA sequencing, the method comprising the steps of:
i) forming eight classes of polynucleotides by extending from primers a plurality of polynucleotides by means of a DNA polymerase or a reverse transcriptase in the presence of a dideoxyadenosine triphosphate, a dideoxycytosine triphosphate, a dideoxyguanosine triphosphate, and a dideoxythymidine triphosphate, and wherein the eight classes of polynucleotides are labeled at a 5′ position with a different fluorophore;
ii) electrophoretically separating the classes of polynucleotides;
iii) illuminating the separated polynucleotides with a wavelength capable of causing the fluorophores to fluoresce; and
iv) identifying the classes of polynucleotides in the bands by the fluorescence or absorption spectrum of the fluorophores.

14. The method of claim 13, wherein said fluorophores comprise at least one BODIPY fluorophore selected from the group consisting of BODIPY 542/563, BODIPY B410, BODIPY B411, BODIPY 503/512, BODIPY 523/547, BODIPY 581/591, BODIPY 630/650 and BODIPY 650/665.

15. The method of claim 13, wherein said fluorophores are selected from the group consisting of fluoresceins, rhodamines, cyanines, coumarins, sulfonated pyrenes, squaraines and alexas.

16. The method of claim 13, wherein each of the fluorophores exhibits a characteristic adsorption maxima that is spectrally resolved as compared to the other fluorophores in the set, and the adsorption maxima is in the range of about 500 to about 700 nm.

17. The method of claim 13, wherein the DNA polymerase is Thermosequenase, AmpliTaqFS, Klenow fragment, SEQUENASE® DNA polymerase, Bst DNA polymerase, AMPLITAQ® DNA polymerase, Pfu (exo-)DNA polymerase, rTth DNA polymerase or Vent(exo-) DNA polymerase.

18. The method of claim 13, wherein the reverse transcriptase is AMV-RT, M-MuLV-RT or SuperScript RT.

19. The method of claim 13, wherein each BODIPY fluorophore in the set is coupled to the primer suitable for sequencing by a linker.

20. The method of claim 13, wherein each BODIPY fluorophore in the set is attached at the 5′ end of the products of the sequencing reaction and an additional fluorophore is attached at a 3′ position of the product of the sequencing reaction or at one or more internal positions of the products of the sequencing reaction.

21. The method of claim 13, wherein the additional fluorophore has an adsorption maxima of about 500 to about 700 nm and an emission maxima of about 500 to about 700 nm.

22. The method of claim 13, wherein the chain termination method of sequencing is performed by an automated DNA sequencing instrument.

23. A method for distinguishing polynucleotides having different ribonucleotides in a method of labeling polynucleotides by enzymatic incorporation, said method comprising the steps of:
i) forming a mixture of four classes of polynucleotides, the four classes comprising polynucleotides having different terminal nucleotide triphosphates, wherein said triphosphates are linked to a BODIPY fluorophore that contains at least one reactive functional group; and wherein said BODIPY fluorophores comprise a first set and all are different;
ii) forming a second mixture of four classes of polynucleotides, the four classes comprising polynucleotides having different terminal nucleotide triphosphates; wherein said triphosphates are linked to a BODIPY fluorophore that contains at least one reactive functional group; and wherein said BODIPY fluorophores comprise a second set and all are different, and are different than the first set;
iii) electrophoretically separating the polynucleotides in the first mixture and the second mixture;
iv) illuminating the first and second mixtures with a wavelength capable of causing the fluorophores to fluoresce; and
v) identifying the classes of polynucleotides in the bands by the fluorescence or absorption spectrum of the fluorophores.

24. The method of claim 23, wherein said terminal nucleotide triphosphates are adenosine triphosphate, guanosine triphosphate, cytidine triphosphate, and uridine triphosphate.

25. The method of claim 23, wherein said terminal nucleotide triphosphates are deoxyadenosine triphosphate, deoxyguanosine triphosphate, deoxycytidine triphosphate, and deoxythymidine triphosphate.

26. The method of claim 23, wherein said terminal nucleotide triphosphates are dideoxyadenosine triphosphate, dideoxyguanosine triphosphate, dideoxycytidine triphosphate, and dideoxythymidine triphosphate.

27. The method of claim 23, wherein said first set comprises at least one fluorophore selected from the group consisting of BODIPY 542/563, BODIPY B410, BODIPY B411, BODIPY 503/512, BODIPY 523/547, BODIPY 581/591, BODIPY 630/650 and BODIPY 650/665.

28. A method of labeling a nucleic acid for 8-color sequencing comprising the steps of:
i) forming a plurality of oligonucleotides substituted with at least two fluorophores comprising a donor and an acceptor; wherein said oligonucleotides are separated into eight classes, wherein said eight donor fluorophores comprise a donor set and are the same or different; and wherein eight acceptor fluorophores comprise an acceptor set and are all different;
ii) annealing said oligonucleotide classes to a strand of a polymerase chain reaction product to generate a substrate for a 5′ to 3′ exonuclease activity;
iii) amplifying said oligonucleotide classes, wherein said exonuclease activity degrades said oligonucleotides, wherein said donor is released; and
iv) detecting said oligonucleotide classes.

29. The method of claim 28, wherein the acceptor fluorophore is different in all eight classes of oligonucleotides and the donor fluorophore is different or the same.

30. The method of claim 28, wherein said acceptor set and donor set comprise at least one BODIPY fluorophore selected from the group consisting of BODIPY 542/563, BODIPY B410, BODIPY B411, BODIPY 503/512, BODIPY 523/547, BODIPY 581/591, BODIPY 630/650 and BODIPY 650/665.

31. As a composition of matter, a 4,4-difluoro-5,7-dimethyl-4-bora-3a,4a-diaza-s-indacene-3-styryloxyacetate.

32. As a composition of matter, a 4,4-difluoro-5-phenyl-4-bora-3a,4a-diaza-s-indacene-3-styryloxyacetate.

33. As a composition of matter, a 4,4-difluoro-5-(4-methoxyphenyl)-4-bora-3a,4a-diaza-s-indacene-3-propionic acid.
